SEQUENCE LISTING (9 sequences)

SEQ ID NO: 1 - protein, 972 aa, Infectious pancreatic necrosis virus
MSTSKATATYLRSIMLPENGPASIPDDITERHILKQETSSYNLEVSESGSGLLVCFPGAPGSRVGAHYRWNLNQTALEFDQWLETSQDLKKAFNYGRLISRKYDIQSSTLPAGLYALNGTLNAATFEGSLSEVESLTYNSLMSLTTNPQDKVNNQLVTKGITVLNLPTGFDKPYVRLEDETPQGPQTMNGARMRCTAAIAPRRYEIDLPSERLPTVAATGTPTTIYEGNADIVNSTTVTGDITFQLEAEPVNETRFDFILQFLGLDNDVPVVTVTSSTLVTADNHRGASAKFTQSIPTEMITKPITRVKLAYQLNQQTAIANAATLGAKGPASVSFSSGNGNVPGVLRPITLVAYEKMTPQSILTVAGVSNYELIPNPDLLKNMVTKYGKYDPEGLNYAKMILSHREELDIRTVWRTEEYKERTRAFTEITDFTSDLPTSKAWGWRDLVRGIRKVAAPVLSTLFPMAAPLIGAADQFIGDLTKTNSAGGRYLSHAAGGRYHDVMDSWASGSEAGSYSKHLKTRLESNNYEEVELPKPTKGVIFPVVHTVESAPGEAFGSLVVVIPGAYPELLDPNQQVLSYFKNDTGCVWGIGEDIPFEGDDMCYTALPLKEIKRNGNIVVEKIFAGPAMGPSSQLALSLLVNDIDEGIPRMVFTGEIADDEETVIPICGVDIKAIAAHEHGLPLIGCQPGVDEMVANTSLASHLIQSGALPVQKAQGACRRIKYLGQLMRTTASGMDAELQGLLQATMARAKEVKDAEVFKLLKLMSWTRKNDLTDHMYEWSKEDPDAIKFGRLVSTPPKHQEKPKGPDQHTAQEAKATKISLDAVKAGADFASPEWIAENNYRGPAPGQFKYYMITGRVPNPGEEYEDYVRKPITRPTDMDKIRRLANSVYGLPHQEPAPDDFYQEVVEVFAENGGRGPDQDQMQDLRDLARQMKRRPRPAETRRQTKTPPRAATSGGSRFTPSGDDGEV

SEQ ID NO: 2 - DNA, 3053 bp, Infectious pancreatic necrosis virus
agcggagagctcttacggaggagctctccgtcgatggcgaaagccctttctaacaaacaaccaacaattcttatctacatgaatcatgagcacatccaaggcaaccgcaacctacttgagatccattatgcttcccgagaatgggccagcaagcattccggacgacataacagagaggcatatactaaaacaagagacctcgtcatacaacttagaggtatccgaatcaggaagtgggcttcttgtctgcttccctggagctcctggatccagggtcggtgcccactacaggtggaatctgaaccagacggcactagaattcgaccagtggctagagacgtcacaggacctaaagaaggcattcaactacgggagactgatctcacggaaatacgacatccagagctcaacccttcccgctggtctgtatgcactcaatgggaccctgaacgctgccaccttcgaaggaagtctgtctgaagtagagagcctaacctacaacagcttgatgtccctaacaacaaacccacaggacaaggtcaacaatcaactagtgaccaaaggaattaccgtcctgaatctaccaactgggtttgacaagccatacgtccgcctagaggacgagacgccacagggcccccagaccatgaacggagcaaggatgaggtgcacagctgccatcgcaccaaggaggtacgaaatcgacctcccatccgaacgactgccgaccgtggccgcgactgggaccccaacaacaatctatgaagggaatgctgacatcgtgaactccacaacagtcaccggggacataacattccagctcgaggccgaacccgtcaatgagacacggttcgacttcatcctacagttcctgggactggacaacgacgtccccgtggttaccgtgacaagctccacgctagtcacagcggacaaccacaggggggcgtcagccaagttcacccagtcaatcccaacagaaatgattaccaaaccaatcacacgggtcaagctggcctaccagctcaaccagcagaccgcaattgcaaacgcagcaacgctcggagccaaggggccggcatcagtctcattctcatccgggaacggcaatgtaccgggggtcctaagacccataaccctagtggcgtacgagaagatgaccccccagtcaatcctgaccgtggctggcgtatccaactatgagctgatcccaaacccagacctactgaagaacatggtcaccaagtatggaaagtatgaccctgagggcctcaactatgccaagatgatcctgtcccacagagaggagctggacatcagaaccgtctggaggactgaggaatacaaagaaaggacaagagcattcacagagatcactgacttcacaagtgacctaccaacctcaaaggcatggggatggagggacctggtcagaggcatcagaaaagtggccgcccccgtgctgtcaacgctcttcccaatggcggctcccctcataggagctgccgaccaattcattggggacctcaccaagaccaactcagccgggggacgctacctgtcacacgcagccggaggccgctaccatgatgtcatggactcatgggccagcggatccgaagcaggaagctactcaaagcacctcaagacccggcttgagtccaataactatgaggaagtggagcttccaaagccaacaaagggagtcatctttcctgtggtgcacaccgttgagagtgcaccaggtgaggcctttgggtcgctcgtggtagtaataccgggagcgtacccggaacttcttgacccaaaccaacaggtcctatcctacttcaagaacgacacaggttgcgtctgggggataggagaagacataccctttgaaggagatgacatgtgctacaccgcactgcccctcaaggagatcaagaggaacggaaacatcgtggtggagaaaatattcgctggccccgcgatgggaccgtcctcccaacttgcactgtccctgctcgtcaacgacatagacgaagggattccaaggatggtcttcacaggcgagattgctgatgacgaagaaacagtcatcccaatctgcggagtggacatcaaagccatagccgcccatgaacatgggctgcccctcattggctgtcagccaggggtcgacgagatggtggcaaacacatctctcgcatcacacctgattcagagcggggccctaccagtgcagaaagcacagggcgcctgcaggagaatcaagtacctgggccagctgatgagaacaactgcatcagggatggacgcagaactgcaggggctgctgcaggccaccatggccagagcaaaagaagtgaaggacgccgaagtgttcaaacttctgaaactcatgtcatggacaagaaagaacgacctcacagatcacatgtatgagtggtcaaaggaggatcctgatgcaatcaaatttggcaggctcgtcagcacccccccaaaacaccaagagaagccaaaaggacctgaccagcacaccgcccaggaggcaaaggccaccaagatctcactggacgccgtcaaagccggcgcagactttgcctccccagagtggatcgcggagaacaactaccgcggcccagccccaggccagttcaagtactacatgataacgggcagagtcccaaaccccggagaagagtacgaggactacgtgcggaaaccgataacccgaccaaccgacatggacaaaatcagacgcctagccaacagtgtctatggcctgccccaccaagaacccgcaccagacgacttctaccaggaagtcgtcgaggtgttcgcagaaaacgggggaagagggcccgaccaagaccaaatgcaagacctgagggacttggcaaggcagatgaaacgacgaccccgaccagctgagacacgcaggcaaactaagactccacccagggcggcaacctccggtggatcgcggtttaccccctccggcgatgacggagaagtgtaacagctactctctttcctgactgatcccctggcctcaaccccggcccccc

SEQ ID NO: 3 - protein, 845 aa, Infectious pancreatic necrosis virus
MSDIFNSPQNKASILSALMKSTAGDVEDVLIPKRFRPAKDPLDSPQAAAQFLKDNKYRILRPRAIPTMVELETDAALPRLRQMVDDGKLKDTVSVPEGTTAFYPKYYPFHKPDHDEVGTFGAPDITLLKQLTFFLLENDFPTGPETLRQVREAIATLQYGSGSYSGQLNRLLAMKGVATGRNPNKTPKTVGYTNEQLAKLLEQTLPINTPKHEDPDLRWAPSWLINYTGDLSTDKSYLPHVTIKSSAGLPYIGKTKGDTTAEALVLADSFIRDLGKAATSADPEAGVKKTITDFWYLSCGLLFPKGERYTQIDWDKKTRNIWSAPYPTHLLLSMVSSPVMDESKLNITNTQTPSLYGFSPFHGGMDRIMTIIRDSLDNDEDLVMIYADNIYILQDNTWYSIDLEKGEANCTPQHMQAMMYYLLTRGWTNEDGSPRYNPTWATFAMNVAPSMVVDSSCLLMNLQLKTYGQGSGNAFTFLNNHLMSTIVVAEWVKAGKPNPMTKEFMDLEEKTGINFKIERELKNLKETIIEAVETAPQDGYLADGSDLPPHRPGKAVELDLLGWSAIYSRQMEMFVPVLENERLIASAAYPKGLENKTLARKPGAEIAYQIVRYEAIRMVGGWNNPLLETAAKHMSLDKRKRLEVKGIDVTGFLDDWNNMSEFGGDLEGITLSEPLTNQTLIDINTPLESFDPKTRPQTPRSPKKTLDEVTAAITSGTYKDPKSAVWRLLDQRTKLRVSTLRDQASALKPASSSVDNWAEATEELAEQQQLLMKANNLLKSSLTETREALETIQSDKIIAGKSNPEKNPGTAANPVVGYGEFSEKIPLTPTQKKNAKRREKQRRNQ

SEQ ID NO: 4 - DNA, 2779 bp, Infectious pancreatic necrosis virus
ccggatgttgtctggggtcatgttggtggcacgttgtcgggtcactacttttgttcagcttgccgtgcggcgcggagcttgcatatgagtccagctgggtcatttcggattctccccggtttggtcttctcactggtttcgtctctgcttctccctccgcttggcattcttcttttgcgtgggagtcagaggaatcttttcgctgaattccccgtagccaaccactgggttggctgctgttcccgggttcttttcgggattggattttccagcgatgattttgtcggactggatggtttccagtgcttctctggtctccgtcaggctgctcttcagcaggttgttggccttcatgagaagttgttgctgctccgctagttcctctgtggcttcggcccagttgtctactgaggatgatgccggtttcagcgccgaggcctgatctcgcagtgtgctgacccggagtttggttctttggtcgagcagtcgccacactgcgctcttggggtccttgtaggtccccgatgttatggcggctgttacctcgtctagggttttctttggagacctgggtgtttgtggtctcgtctttgggtcgaagctctccagtggtgtgttgatgtctatcagggtttggtttgtgaggggttcggatagtgtgattccttctaggtctcctccgaactcggacatgttgttccagtcgtcgaggaaccctgtcacgtcgatccccttcacctccagtctcttcctcttgtcgagggacatgtgttttgctgcggtttccagcagtggattgttccagccgcccaccatcctgattgcttcgtaccttactatttgatatgcaatctcggccccgggtttccgggcaagggtcttgttctcaagcccttttgggtaggcggctgatgcaattagtcgttcgttctcgaggactgggacgaacatctccatttggcggctgtagatggccgaccagcccagcaggtcaagctctacggcttttcctggcctgtgcggtggtagatcggagccatcggcaaggtagccatcctggggagccgtttccacggcctcgatgatggtctccttgaggttctttagctctcgctcaatcttgaagttgatccccgtcttttcttcgaggtccatgaactcttttgtcatggggtttggttttcctgccttcacccactcagccacgacgatcgtggacatgaggtgattgttcaggaaggtaaaggcgttcccactgccctggccgtaggtcttcagctgaagattcattagcaggcaggatgagtccacgaccatcgacggtgcaacgttcatggcgaaggttgcccacgtcgggttgtaccgtggagagccgtcctcattcgtccacccccttgtcaggaggtagtacatcatggcctgcatgtgttgtggggtgcagttggcctcgcccttctccaggtctatcgagtaccacgtgttgtcttgcaggatgtagatgttgtctgcatatatcatcactaggtcctcgtcgttgtccaggctgtctctgatgattgtcatgatcctgtccatccctccgtggaatggtgagaacccgtacagagatggggtctgggtgttggtgatgttgagttttgactcgtccatcacaggggacgacaccattgatagtagtaggtgtgttgggtagggcgcactccagatgtttctggtcttcttgtcccagtcgatctgtgtgtatctctcgccctttgggaatagcagcccacagcttagataccagaagtcggtgattgtcttcttcaccccagcttctgggtccgctgatgttgctgcttttccaaggtcacgtatgaaggagtcagccagtacgagcgcttcggcagtcgtgtctcctttggttttgcctatgtacggtaggccggctgaggactttatggtcacatgtggcaggtatgacttgtctgtgctcaggtctccggtgtagttgatcaaccagctgggggcccaccggaggtcggggtcctcatgttttggagtgttgatcggtagggtttgctccagcagtttggccagctgctcgttggtgtagcctactgtttttggagtcttgtttggattcctgccagtagcgactcctttcatggctaggagcctgttcagttgtccggagtagctgcctgagccatactggagtgtggctatggcctcgcggacttgcctgagtgtctctggtcctgtggggaagtcattttccaacaggaagaaggtgagttgcttcagaagtgtgatgtccggagccccgaacgtcccaacttcgtcatgatctggcttgtggaatgggtagtattttgggtaaaatgcagtggttccctctgggacgcttaccgtgtccttgagcttgccatcgtctaccatttgtcgcagtcgaggcagagcggcatctgtctctagttcgaccatggtcggaatggctcgcggcctaagtatccgatacttgttgtcctttaggaactgtgctgcggcttgcgggctgtcgaggggatccttggctggcctgaagcgcttgggtatcagcacgtcttctacatctcctgctgtgctcttcatgagagcgctcaagatagaggccttgttctgtggtgagttgaagatgtccgacatatcatgagttgtgtgtatagagatttgttaggggcacttgttgtcctttaggaactgtgctgcggcttgcgggctgtcgaggggatccttggctggcctgaagcgcttgggt

SEQ ID NO: 5 - protein, 1012 aa, Infectious bursal disease virus
MTNLQDQTQQIVPFIRSLLMPTTGPASIPDDTLEKHTLRSETSTYNLTVGDTGSGLIVFFPGFPGSIVGAHYTLQSNGNYKFDQMLLTAQNLPASYNYCRLVSRSLTVRSSTLPGGVYALNGTINAVTFQGSLSELTDVSYNGLMSATANINDKIGNVLVGEGVTVLSLPTSYDLGYVRLGDPIPAIGLDPKMVATCDSSDRPRVYTITAADDYQFSSQYQAGGVTITLFSANIDAITSLSIGGELVFQTSVQGLILGATIYLIGFDGTAVITRAVAADNGLTAGTDNLMPFNIVIPTSEITQPITSIKLEIVTSKSGGQAGDQMSWSASGSLAVTIHGGNYPGALRPVTLVAYERVATGSVVTVAGVSNFELIPNPELAKNLVTEYGRFDPGAMNYTKLILSERDRLGIKTVWPTREYTDFREYFMEVADLNSPLKIAGAFGFKDIIRALRRIAVPVVSTLFPPAAPLAHAIGEGVDYLLGDEAQAASGTARAASGKARAASGRIRQLTLAADKGYEVVANLFQVPQNPVVDGILASPGILRGAHNLDCVLREGATLFPVVITTVEDAMTPKALNSKMFAVIEGVREDLQPPSQRGSFIRTLSGHRVYGYAPDGVLPLETGRVYTVVPIDGVWDDSIMLSKDPIPPIVGSSGNLAIAYMDVFRPKVPIHVAMTGALNAYGEIENVSFRSTKLATAHRLGLKLAGPGAFDVNTGSNWATFIKRFPHNPRDWDRLPYLNLPYLPPNAGRQYDLAMAASEFKETPELESAVRAMEAAANVDPLFQSALSVFMWLEENGIVTDMANFALSDPNAHRMRNFLANAPQAGSKSQRAKYGTAGYGVEARGPTPEGAQREKDTRISKKMETMGIYFATPEWVALNGHRGPSPGQLKYWQNTREIPDPNEDYLDYVHAEKSRLASEGQILRAATSIYGAPGQAEPPQAFIDEVAKVYEVNHGRGPNQEQMKDLLLTAMEMKHRNPRRAPPKPKPKPNVPTQRPPGRLGRWIRAVSDEDLE

SEQ ID NO: 6 - DNA, 3183 bp, Infectious bursal disease virus
gatacgatcggtctgaccccgggggagtcaccctgggacaggctggcaaggccttgttccaggatggaactcctccttctacaatgctatcattgatggttagtagagatcagacaaacgatcgcagcgatgacaaacctgcaagatcaaacccaacagattgttccgttcatacggagccttctgatgccaacaaccggaccggcgtccattccggacgacaccctagagaagcacactctcaggtcagagacctcgacctacaatttgactgtgggggacacagggtcagggctaattgtctttttccctggcttccctggctcaattgtgggtgctcactacacactgcagagcaatgggaactacaagttcgatcagatgctcctgactgcccagaacctaccggccagctacaactactgcaggctagtgagtcggagtctcacagtgaggtcaagcacactccctggtggcgtttatgcactaaatggcaccataaacgccgtgaccttccaaggaagcctgagtgaactgacagatgttagctacaatgggttgatgtctgcaacagccaacatcaacgacaaaatcgggaacgtcctagtaggggaaggggtaaccgtcctcagcttacccacatcatatgatcttgggtatgtgagactcggtgaccccattcccgctatagggctcgacccaaaaatggtagcaacatgtgacagcagtgacaggcccagagtctacaccataactgcagccgacgattaccaattctcatcacagtaccaagcaggtggggtaacaatcacactgttctcagctaatatcgatgccatcacaagcctcagcatcgggggagaactcgtgtttcaaacaagcgtccaaggccttatactgggtgctaccatctaccttataggctttgatgggactgcggtaatcaccagagctgtggccgcagacaatgggctgacggccggcactgacaaccttatgccattcaatattgtgattccaaccagcgagataacccagccaatcacatccatcaaactggagatagtaacctccaaaagtggtggtcaggcgggggatcagatgtcatggtcagcaagtgggagcctagcagtgacgatccacggtggcaactatccaggggccctccgtcccgtcacactagtagcctacgaaagagtggcaacaggatctgtcgttacggtcgccggggtgagcaacttcgagctgatcccaaatcctgaactagcaaagaacctggtcacagaatacggccgatttgacccaggagccatgaactacacaaaattgatactgagtgagagggaccgtcttggcatcaagaccgtatggccaacaagggagtacactgactttcgcgagtacttcatggaggtggccgacctcaactctcccctgaagattgcaggagcatttggcttcaaagacataatccgggccctaaggaggatagctgtgccggtggtctctacactgttcccacccgccgctcccctagcccatgcaattggggaaggtgtagactacctgctgggcgatgaggcacaggctgcttcaggaactgctcgagccgcgtcaggaaaagcaagagctgcctcaggccgcataaggcagctaactctcgccgccgacaaggggtacgaggtagtcgcgaatctgttccaggtgccccagaatcctgtagtcgacgggattctcgcttcacccgggatactccgcggtgcacacaacctcgactgcgtgttgagagagggtgccacgctattccctgtggtcatcacgacagtggaagatgccatgacacccaaagcactgaacagcaaaatgtttgctgtcattgaaggcgtgcgagaagatctccaacctccatctcaaagaggatccttcatacgaactctctccggacatagagtctatggatatgctccagatggggtacttccactggagactgggagagtttacaccgtggtcccaatagatggtgtctgggacgacagcattatgctgtccaaagaccccatacctcctattgtgggaagcagcggaaacctagccatagcttacatggatgtgtttcgacccaaagtccccatccatgtggccatgacaggagccctcaacgcctatggcgagattgagaatgtgagctttagaagcaccaagctcgccactgcacaccgacttggcctcaagttggctggtcccggtgcatttgacgtgaacaccgggtccaactgggcgacgtttatcaaacgttttcctcacaatccacgcgactgggacaggctcccctacctcaaccttccataccttccacccaatgcaggacgccagtacgacctggccatggccgcttcagagttcaaagagacccccgaactcgagagcgccgtcagagccatggaagcagcagccaacgtggacccactgttccaatctgcgctcagcgtgttcatgtggctggaagagaatgggattgtgactgatatggccaacttcgcactcagcgacccgaacgcccatcggatgcgcaattttctcgcaaacgcaccacaagcaggcagcaagtcgcaaagagccaagtacgggacagcaggctacggagtggaggcccggggccccactccagagggagcacagagggaaaaagacacacggatctcaaagaagatggagactatgggcatctactttgcaacaccagaatgggtagcactcaatgggcaccgggggccaagccccggccagctgaagtactggcagaacacacgagaaatacctgatccaaacgaggactacctagactacgtgcatgcagagaagagccggttggcgtcagaaggacaaatcctaagggcagctacgtcgatctacggggctccaggacaggcagagccaccccaagccttcatagacgaagtcgccaaagtctatgaagtcaaccatgggcgtggccccaaccaagaacagatgaaagatctgctcttgactgcgatggagatgaagcatcgcaatcccaggcgggccccaccaaagcccaagccaaaacccaatgttccaacacagagaccccctggtcggttgggccgctggatcagggctgtctctgatgaggaccttgagtgaggctcctgggagtct

SEQ ID NO: 7 - protein, 879 aa, Infectious bursal disease virus
MSDVFNSPQARSKISAAFGIKPTAGQDVEELLIPKVWVPPEDPLASPSRLAKFLRENGYKILQPRSLPENEEYETDQILPDLAWMRQIEGPVLKPTLSLPIGDQEYFPKYYPTHRPSKEKPNAYPPDIALLKQMIYLFLQVPEATDNLKDEVTLLTQNIRDKAYGSGTYMGQATRLVAMKEVATGRNPNKDPLKLGYTFESIAQLLDITLPVGPPGEDDKPWVPLTRVPSRMLVLTGDVDGEFEVEDYLPKINLKSSSGLPYVGRTKGETIGEMIAISNQFLRELSALLKQGAGTKGSNKKKLLSMLSDYWYLSCGLLFPKAERYDKSTCCTKTRNKWSAQSSTHLMISMITWPVMSNSPNNVLNIEGCPSLYKFNPFRGGLNRIVEWIMAPDEPKALVYADNIYIVHSNTWYSIDLEKGEANCTRQHMQAAMYYILTRGWSDNGDPMFNQTWATFAMNIAPALVVDSSCLIMNLQIKTYGQGSGNAATFINNHLLSTLVLDQWNLMKQPSPDSEEFKSIEDKLGINFKIERSIDDIRGKLRQLVPLAQPGYLSGGVEPEQPSPTVELDLLGWSATYSKDLGIYVPVLDKERLFCSAAYPKGVENKSLKSKVGIEQAYKVVRYEALRLVGGWNYPLLNKACKNNASAARRHLEAKGFPLDEFLAEWSELSEFGEAFEGFNIKLTVTPESLAELNRPVPPKPPNVNRPVNTGGLKAVSNALKTGRYRNEAGLSGLVLLATARSRLQDAVKAMAEAEKLHKSKPDDPDADWFERSETLSDLLEKADIASKVAHSALVETSDALEAVQSTSVYTPKYPEVKNPQTASHPVVGLHLPAKRATGVQAALLGAGTSRPMGMEAPTRSKNAVKMAKRRQRQKESRQ

SEQ ID NO: 8 - DNA, 2715 bp, Infectious bursal disease virus
agctgctacggtaagcgtccctcttcttgatgattctaccaccatgagtgacgttttcaatagtccacaggcgcgaagcaagatatcagcagcgttcggcataaagcctacagctggacaagatgtggaagaactcctgatccctaaggtctgggtgccacctgaggatcccttggccagccctagtcgtctggccaagttcctcagggaaaacggctacaagattctgcagccacggtctctacctgagaatgaggagtatgagaccgatcaaatactccctgacctagcttggatgaggcagatagagggacctgttttaaaaccaaccctatctctccccattggggaccaggagtacttcccaaaatactacccaacacaccgcccgagcaaggaaaagcccaatgcgtacccgcccgatatcgcattactcaagcagatgatctacttgtttctccaggttcccgaggccacagataacctcaaagatgaggtcaccctactaacccaaaacattagagataaagcctacgggagtgggacctacatgggacaggccaccagacttgttgctatgaaagaggttgccactgggagaaacccaaacaaagatcctctaaagcttgggtacacctttgagagcatagcccagctacttgacatcactttaccggtaggcccacccggtgaggatgacaagccctgggtaccactcacaagggtgccgtcaaggatgttggttctgacgggcgacgtagatggggaatttgaggttgaggactaccttcccaaaatcaacctcaagtcatcaagtggactgccctatgttggtcgcaccaaaggagaaactattggggagatgatagccatatcgaaccagtttcttcgagagctatcagcgctgctgaagcagggtgcagggacaaaagggtcgaacaagaagaagctgctcagcatgctaagtgactactggtacttatcatgtgggcttttgtttcccaaggctgagaggtacgacaaaagcacatgctgcaccaagacccgtaacaaatggtcagctcaatcgtcaacacacctgatgatctcaatgataacctggcccgtgatgtccaatagcccaaacaacgtgttgaacattgaggggtgtccgtcactctacaagttcaacccgtttagaggtgggctaaacaggatcgtggagtggataatggctccggatgaacccaaggccttagtatatgctgacaacatatacattgttcactccaacacgtggtactcaattgacctagagaagggtgaggcaaactgcacgcgtcaacacatgcaggccgccatgtactacatccttaccagaggatggtccgataacggtgaccccatgttcaatcagacatgggccacctttgcgatgaacattgccccagccctagttgtggactcatcatgtctgattatgaaccttcagatcaagacatatggtcaaggcagtgggaatgcagccaccttcatcaacaaccatcttcttagcacccttgtgctagaccagtggaacttgatgaagcaacctagtccagacagcgaagagttcaagtcaattgaagacaagctgggcatcaacttcaagattgagaggtccattgatgacattaggggcaagctcagacagcttgtcccccttgcacaaccagggtacctgagtggaggggtcgaaccagagcaacccagcccaactgtagagctggacctactcggatggtctgcaacttacagcaaagatcttgggatctatgtgccggtgcttgacaaggaacgcttattttgctctgctgcgtatcccaaaggggtagagaataaaagcctcaaatccaaggttgggatcgagcaagcatacaaagttgtcaggtacgaggcgttgaggttggtaggtggttggaactacccactcctgaacaaagcttgcaagaacaatgcaagtgcagctcggcggcatctggaggccaaggggttcccgctcgatgagttcctcgccgagtggtcagagttgtccgagttcggagaagctttcgaaggcttcaacatcaagctgacagtaacaccggagagcctcgccgaacttaacagaccagtaccccccaaacctccaaatgtcaacagaccagtcaacaccggtgggctaaaggcagtcagcaatgccctcaagaccggtcggtatagaaatgaagccggactaagtggcctcgtcctcctagccaccgcccgcagccgactacaggacgctgtcaaggccatggcagaggccgagaaactccacaagtctaagcccgatgaccccgatgcagactggtttgaacgatcagaaaccctgtcagacctgctggagaaagccgacattgccagcaaggtcgctcactcagcactcgtggaaacaagcgacgctcttgaagcggtccagtcaacctcagtgtacaccccaaagtacccagaggttaagaacccacagaccgcctcccaccccgttgttgggctccacctgcccgccaagagggccaccggtgtccaggcagctcttctcggagcagggacgagcagaccaatggggatggaggccccaacacggtccaagaacgccgtaaaaatggccaaaaggcggcaacgccaaaaagagagccgccaatagccatgatgggaaccattcaagaagaggacact

SEQ ID NO: 9 - DNA, 229 bp, artificial sequence
  engineered VP5 mutant coding sequence
agtggtaacccacgagcggagagctcttacggaggagctctccgtcgatggcgaaagccctttctaacaaacaaccaacaattcttatctacaggaatcatgagcacatccaaggcaaccgcaacctacttgagatccattatgcttcccgagaatgggccagcaagcattccggatgacataacagagaggcatatactaaaacaagagacctcgtcatacaacttag

(19) United States
(12) Patent Application Publication
(10) Pub. No.: US 2024/0245739 A1
(43) Pub. Date: Jul. 25, 2024

(54) ONCOLYTIC VIRUS BASED CANCER THERAPY

(71) Applicant: The Penn State Research Foundation, University Park, PA (US)

(72) Inventor: Suresh KUCHIPUDI, State College, PA (US)

(21) Appl. No.: 18/562,068

(22) PCT Filed: May 17, 2022
(86) PCT No.: PCT/US2022/029612

§ 371 Date: Nov. 17, 2023

Related U.S. Application Data
(60) Provisional application No. 63/189,834, filed on May 18, 2021.

(51) Int. Cl. A61K 35/768; A61P 35/00; C12N 7/00

(52) U.S. Cl. CPC A61K 35/768; A61P 35/00; C12N 7/00; C12N 2720/10021; C12N 2720/10032; C12N 2720/10043

(57) ABSTRACT

Provided are compositions and methods for prophylaxis or therapy of cancer. The compositions and methods include use of Birnaviridae double stranded RNS (dsRNA) viruses for treating cancer in individuals that are not the normal virus hosts. Modifications of the dsRNA viruses are provided and include genetic changes that result improvements in anti-cancer therapy by increasing onxotoxicity of the modified viruses, and by including sequences encoding therapeutic pay loads.

### CROSS REFERENCE TO RELATED APPLICATIONS

This application claims priority to U.S. provisional patent application No. 63/189,834, filed May 18, 2021, the entire disclosure of which is incorporated herein by reference.

### STATEMENT REGARDING FEDERALLY SPONSORED RESEARCH

This invention was made with government support under Hatch Act Project No. PEN04588 awarded by the United States Department of Agriculture/NIFA. The government has certain rights in the invention.

### SEQUENCE LISTING

The instant application contains a Sequence Listing which has been submitted electronically in ASCII format and is hereby incorporated by reference in its entirety. Said ASCII file, created on May 11, 2022, is named PSU_2019_400_sequence_ST25.txt, and is 48,400 byte in size.

### FIELD

The present disclosure relates generally to compositions and methods for treating cancer, and more specifically to use of double stranded (ds) RNA viruses and modified versions thereof for use as oncolytic virotherapy.

### BACKGROUND

Oncolytic virotherapy is an emerging cancer treatment modality which uses replication competent viruses to destroy cancers. An oncolytic virus (OV) is a genetically engineered or naturally occurring virus that can selectively replicate in and kill cancer cells without harming the normal tissues¹. The ability to selectively infect only cancer cells and not healthy cells (Oncoselectivity) and the ability to effectively infect and kill cancer cells (Oncotoxicity) are key characteristics of ideal OVs. Consequently, several animal viruses that are nonpathogenic to humans such as the Newcastle Disease virus (NDV) and vesicular stomatitis virus (VSV) are useful in developing Oncolytic virotherapy²⁻¹⁴. Many animal viruses cannot normally replicate in healthy human cells, however, cancer cells have impaired antiviral responses induced by type I interferon pathways and make them susceptible to animal viruses like VSV⁸. Currently it is believed there is only one oncolytic virus therapy approved by the FDA for the treatment of cancer, which is a modified herpes simplex virus (HSV) that infects and promotes killing of melanoma tumor cells. There is an ongoing and unmet need for new OVs and methods of using them for selective targeting of cancer cells. The present disclosure is pertinent to this need.

### BRIEF SUMMARY

The present disclosure provides compositions and methods for treating cancer. The compositions comprise isolated or recombinantly produced oncolytic double stranded RNA (dsRNA) virus (referred to herein as “OVs”). The OVs are either a wild type or genetically modified Birnaviridae aquabirnavirus, such as Infectious Pancreatic Necrosis virus (IPNV) (OV1) or a modified OV1, a wild type or genetically modified Birnaviridae avibirnavirus, such as poultry virus Infectious Bursal disease Virus (IBDV) (OV2) or a modified OV2. The genetic modifications include but are not necessarily limited to a disruption or mutation of a segment of the viral genome that encodes the viral VP5 protein such that the viral VP5 protein is not produced within cells infected with the OV1 or the OV2, and or a sequence encoding a therapeutic payload. Substitutions of genomic segments from one OV to another are also included as examples of genetic modifications. The disclosure provides for use of the OVs for treating any type of cancer in an individual that is not the normal host for the OVs, such as any mammal, including but not necessarily limited to humans and canines.

### BRIEF DESCRIPTION OF THE FIGURES

FIGS. 1A-1D. Data showing OV1 and OV2 are effective against a wide range of cancer cells. The viability of renal adenocarcinoma cells (769P) and mammary ductal carcinoma cells (HCC1187) significantly decreased at 72 hours after infection with OV1 (FIGS. 1A & 1B) or OV2 (FIGS. 1C & 1D). Notably OV1 and OV2 significantly reduced viability of 769P and HCC1187 even at very low doses, i.e., multiplicity of infection (MOI) of 0.001 (* p<0.05, ** p<0.01, *** p<0.001, **** p<0.0001).

FIGS. 2A-2D. Data showing OV1 and OV2 activate apoptotic death of cancer cells. Apoptosis levels as measured by the levels of activated Caspase 3 and 7 in renal adenocarcinoma cells (769P) (MOI≥0.001) and mammary ductal carcinoma cells (HCC1187) (MOI≥0.01) increased significantly at 72 hours after infection with OV1 (FIGS. 2A & 2B) or OV2 (FIGS. 2C & 2D). (* p<0.05, ** p<0.01, *** p<0.001, **** p<0.0001).

FIG. 3. Data showing replication of OV1 and OV2 in 4T1.2 cells. Data represents average of 3 replicates.

FIGS. 4A-4C. Data showing anti-tumor activity of OV1 and OV2 in a murine breast cancer model. In vivo study design (FIG. 4A) Mice (n=10/group) were injected with 4T1.2 tumor cells at day 0. At 19 days post tumor implantation, mice were injected with OV1, OV2 or vehicle. Mice were sacrificed on day 35. The changes in tumor sizes post infection with OV1 and 2 (FIG. 4B), and percentage of live cells in the tumor in all the three groups (FIG. 4C) are shown. In the OV treated groups, percentage change in tumor size was significantly smaller compared to control, and there was a reduction of live cells in the tumor in the OV-treated groups. * p≤0.05, ** p≤0.01.

FIG. 5. Depiction of an approach for testing recombinant OVs and recombinant OVs plus payload gene in mice.

FIGS. 6A-6B. Data showing efficacy of OV2 against canine mammary carcinoma cells (CMT-U27). Results of MTS (3-(4,5-dimethylthiazol-2-yl)-5-(3-carboxymethoxyphenyl)-2-(4-sulfophenyl)-2H-tetrazolium) assays and caspase 3,7-Glo assays of CMT-U27 at 72 hours after infection with OV2 show that the cell viability decreased significantly (MOI≥1) (FIG. 6A), and apoptotic levels increased significantly (MOI≥0.1) (FIG. 6B). * p<0.05, ** p<0.01, *** p<0.001, **** p<0.0001.

FIG. 7. Construct map of synthetic DNA with Wild Type Segment A used to produce recombinant IPNV Δ VP-S, IPNV-GM-CSF and IPNV Δ VP-5-GM-CSF viruses.

FIG. 8. Construct map of synthetic DNA with Segment A with granulocyte monocyte colony stimulating factor (GM-CSF) used to produce recombinant IPNV-GM-CSF and IPNV Δ VP-5-GM-CSF recombinant.

FIG. 9. Construct map of synthetic DNA with Wild type Segment B used to produce recombinant IPNV Δ VP-5, IPNV-GM-CSF and IPNV Δ VP-5-GM-CSF viruses.

### DETAILED DESCRIPTION

Unless defined otherwise herein, all technical and scientific terms used in this disclosure have the same meaning as commonly understood by one of ordinary skill in the art to which this disclosure pertains.

Every numerical range given throughout this specification includes its upper and lower values, as well as every narrower numerical range that falls within it, as if such narrower numerical ranges were all expressly written herein. As used herein, the singular forms “a” “and” and “the” include plural referents unless the context clearly dictates otherwise.

The disclosure includes all polynucleotide and amino acid sequences described herein. Each RNA sequence includes its DNA equivalent, and each DNA sequence includes its RNA equivalent. Complementary and anti-parallel polynucleotide sequences are included. Every nucleotide sequence encoding a polypeptide disclosed herein is encompassed by this disclosure. Amino acids of all protein sequences and all polynucleotide sequences encoding them are also included, including but not limited to sequences included by way of sequence alignments. Sequences from 80.00%-99.99% identical to any sequence (amino acids and nucleotide sequences) of this disclosure are included.

The disclosure includes all polynucleotide and all amino acid sequences that are identified herein by way of a database entry. Such sequences are incorporated herein as they exist in the database on the effective filing date of this application or patent. The disclosure of each reference described herein is incorporated herein.

This disclosure relates in part to the presently described discovery that certain members of a group of animal viruses belonging to the family Birnaviridae exhibit potent oncolytic activity. Specifically, members of Birnaviridae comprise viruses grouped in seven genera which include the genus aquabirnavirus comprising the fish pathogen Infectious Pancreatic Necrosis (IPN) virus (IPNV) and the genus avibirnavirus comprising the poultry virus Infectious Bursal disease Virus (IBDV). These viruses are naturally nonpathogenic to humans, and it is demonstrated herein that they cannot replicate productively and induce cell death of normal human cells. Thus, in embodiments, compositions and methods of this disclosure relate to use of a B. aquabirnavirus or B. avibirnavirus, combinations thereof, and modified versions thereof, in oncolytic cancer therapies. IPN virus and IBD virus are also referred to herein as OV1 and OV2, respectively.

OV1 and OV2 contain polyploid bipartite genomes composed of two segments named A and B. Segment A is the larger of the two genome segments and includes two partially overlapping open reading frames (ORFs). The first ORF encodes the nonessential nonstructural viral protein 5 (VP5). VP5 is involved in the nonlytic egression of virus particles but is shown to be dispensable for virus replication. The second ORF encodes a polyprotein that is cotranslationally autocleaved by the viral protease VP4, generating the precursor pVP2, VP4, and VP3. Segment B, the shorter segment in the IBDV genome, is monocistronic and encodes the viral RNA dependent RNA polymerase (RdRp) termed VP1¹⁵.

OV1 and OV2 have small genomes organized as the two segments described above, and they exhibit cytoplasmic replication without risk of host-cell transformation and lack pre-existing immunity in humans and other mammals. In addition, unlike other previously described OVs, a unique feature of OV1 and OV2 (and other members of their genera) are their double stranded RNA genomes. RNA viruses with double-stranded genomes induce the innate immune response through their genome itself 16. This feature imparts an additional advantage to use of the described viruses as OVs because, in addition to their oncotoxicity as demonstrated herein, they have the potential to induce a robust innate immune response and alleviate local immune suppression in the tumor microenvironment 9.

The OV1 segments A and B are available under GenBank accession numbers MH010544.1 and MH010545.1 respectively.

The amino acid sequence under MH010544.1 is SEQ ID NO:1. A cDNA of the viral genome coding strand that encodes SEQ ID NO: 1 is SEQ ID NO:2.

The amino acid sequence under MH010545.1 is SEQ ID NO:3. A cDNA of the viral genome coding strand that encodes SEQ ID NO:3 is SEQ ID NO:4.

The OV2 segment A complete reference sequence is available under NCBI accession no. NC_004178.1 and segment B is available under NCBI Reference Sequence: NC_004179.1.

The amino acid sequence of NC_004178.1 is SEQ ID NO:5. A cDNA of the viral genome coding strand that encodes SEQ ID NO:5 is SEQ ID NO:6.

The amino acid sequence of NC_004179.1 is SEQ ID NO:7. A cDNA of the viral genome coding strand that encodes SEQ ID NO:7 is SEQ ID NO:8.

The sequences of these database entries are incorporated herein entirety as the exist in the database on the filing date of this application or patent, as are all amino acid sequences encoded by these nucleotide sequences. The described sequences include their RNA equivalents, RNA complementary sequences, and RNA reverse complement sequences.

Data presented in this disclosure demonstrate that OV1 and OV2, which as noted above are naturally occurring viruses that are nonpathogenic to humans, do not replicate productively within or induce cell death of normal human cells. However, the disclosure reveals these viruses can infect a range of human cancer cells, replicate, and induce apoptotic cell death, thereby exhibiting oncoselectivity and oncotoxicity properties. Specifically, the disclosure provides in vitro and in vivo data supporting the use of OV1 and OV2 as OV anti-cancer agents as illustrated by the Examples below, which are not intended to limit the disclosure. Thus, OV1 and OV2 and modified versions thereof are new oncolytic virus therapeutic agents.

The disclosure includes modifications of OV1 and OV2. Such modifications include but are not necessarily limited to changes in the viral genomic sequences that do not adversely affect the oncolytic properties of the viruses, e.g., the changes do not reduce oncoselectivity or oncotoxicity, and instead may increase one or both of these properties.

In embodiments, modifications comprise improving the suitability of the viruses for use in treating cancer in mammals, including but not necessarily in humans, and as such, can function at elevated temperatures relative to the ordinary temperatures of their natural hosts. For example, as noted above, members of the B. aquabirnavirus genus include the IPN virus which is found in cold water fish, such as Chinook salmon. Thus, in embodiments, a modified OV of this disclosure is modified such that it can replicate and retain its oncolytic properties at a temperature that is higher than about 30 degrees Celsius. Specifically, OV2 is naturally found in poultry and as such can replicate in temperatures of about 39.7 degrees Celsius and higher. Without intending to be bound by any particular theory, it is considered that viral temperature sensitivity is controlled at least in part by the RNA-dependent-RNA Polymerase (RdRp) that is typical of most dsRNA viruses. In the presentative members of Birnaviridae described herein segment B of the genome encodes the RdRp. Thus, by substituting a segment B of a dsRNA virus that can replicate in a higher temperature with a segment B of a virus that ordinarily replicates at a lower temperature the disclosure provides for modified, hybrid viruses that can be used in the described methods for warm blooded mammals. Additional modifications of the OVs are described below. The disclosure includes treating cancer in any mammal, including but not necessarily limited to humans, canines, felines, and equine animals.

The disclosure also includes improving the oncolytic function of the described viruses by, for example, selecting for mutations that arise during viral replication, such as during serial passaging of the viruses. For example, viral particles obtained from serial passaging can be tested on any type of cancer cells in vitro and/or using a variety of available animal cancer models to select viruses that have any improved property, including but not necessarily increased tropism for particular type of cancer cells, improved immune cell responses, and any other desirable effect.

In addition to the modifications described below, the disclosure includes modifying the viral genome to co-deliver a therapeutic payload. In an embodiment, the disclosure includes a modification of the viral genome that includes deletion of the viral VP5 gene and insertion of a nucleotide sequence encoding the therapeutic payload. Those skilled in the art will recognize the viral VP5 gene sequence from the described genomic segments. The therapeutic payload may be any therapeutic peptide or protein that can be encoded by the viral genome and expressed in infected cells from RNA produced by the viral RdRp. Representative and non-limiting embodiments of therapeutic payloads include granulocyte-macrophage colony-stimulating factor (GM-CSF) such as human GM-CSF (hGM-CSF) and sodium iodides symporter gene. Additional examples of therapeutic payloads include but are not limited to toxins, anti-angiogenic agents, cytokines and chemokines and/or their cognate receptors, antibodies, a chimeric antigen receptor (CAR), a bispecific antibody, microtubule-destabilizing agents, tumor-associated antigens (TAAs) to further stimulate T cells, dopachrome tautomerase, and the like. In embodiments, one or more OVs described herein are modified to encode a fusogenic glycoprotein, and/or to encode an enzyme that can affect the extracellular matrix, such relaxin and hyaluronidase. In embodiments, one or more OVs described herein are modified to encode or are administered with an antiangiogenic agent such as anti-vascular endothelial growth factor (VEGF) antibody, an example of which is bevacizumab. In embodiments, one or more OVs described herein are modified to encode or are administered with angiostatin. In embodiments, one or more OVs described herein are modified to encode one or a combination of IL-15, IFNγ, CCL5, and MG1-IL-12, TNF-α, IL-2, or fibroblast growth factor 2 (FGF-2).

In embodiments, more than one OV can be combined. Thus, the disclosure includes combinations of different OVs and methods of administering the combinations to an individual in need thereof.

The disclosure includes isolated populations of the described OVs, which may be purified to any desired degree of purity. The disclosure includes compositions and methods for making the OVs. For example, the disclosure includes one or more expression vectors encoding the OV proteins and RNA, making OV viral particles from cells comprising the one or more expression vectors, and separating the OVs from the cells. In vitro cells and cell cultures comprising such expression vectors, and cell culture medium comprising viral particles produced by the cells are included within the scope of this disclosure. The disclosure includes polynucleotides that selectively hybridize to the described viral genomes.

In embodiments, the disclosure provides pharmaceutical formulations comprising one or more types of OVs as described herein. Suitable pharmaceutical compositions can be prepared by mixing one or OVs described herein with a pharmaceutically acceptable additive, such as a pharmaceutically acceptable carrier, diluent or excipient, and suitable such components are well known in the art. Some examples of such carriers, diluents and excipients can be found in: Remington: The Science and Practice of Pharmacy 23rd edition (2020), the disclosure of which is incorporated herein by reference. In embodiments, the pharmaceutical formulation does not comprise a cell culture, or a cell culture media. In embodiments, the pharmaceutical formulation is free from any cell culture media. In embodiments, the pharmaceutical formulation is free from any mammalian cells or mammalian cell culture. In embodiments, the pharmaceutical formulation is free of any fish cells, such as salmon embryo (e.g. CHSE) cells, or fowl cells, such as chicken embryo fibroblasts, or any culture media used to propagate such cells. In embodiments, the pharmaceutical formulation is free of amino acids, including but not limited to non-essential amino acids, and/or is free from Foetal Bovine Serum (FBS). In embodiments, the pharmaceutical formulation is free of Basal Medium Eagle (BME), fetal calf serum (FCS) and newborn calf serum (NBCS). In embodiments, the pharmaceutical formulation comprises an anti-cancer effect amount of one or more additives.

Administration of compositions as described herein can be performed using any suitable route of administration, including but not limited to parenteral, intraperitoneal, intrapulmonary, intra-arterial, intravenous, and intra-tumoral injection.

In embodiments, an effective amount of viral particles is administered to an individual. In embodiments, an effective amount is an amount that is sufficient to achieve at least one of: cancer cell killing, inhibition of tumor growth, recurrence or relapse, or inhibition of metastasis. In embodiments, the number of cancer cells are reduced, or all cancer cells are eradicated from an individual. Those skilled in the art, when given the benefit of the present disclosure, will be able to determine an effective amount of viral particles using established techniques, including but not necessarily limited to use of animal models, wherein representative doses are 10⁶ or 10⁷ pfu/mouse, which can be adjusted based on parameters such as the size of the mouse or other individual being treated, the type and stage of cancer, and the like.

In embodiments, a composition comprising the described OVs is administered to an individual in need thereof. In embodiments, the individual in need has been diagnosed with or is suspected of having any type of cancer. In embodiments, the cancer is comprised by a solid tumor or a hematological malignancy. In embodiments, the cancer is breast cancer, prostate cancer, pancreatic cancer, lung cancer, liver cancer, ovarian cancer, cervical cancer, colon cancer, esophageal cancer, stomach cancer, bladder cancer, brain cancer, testicular cancer, head and neck cancer, melanoma, skin cancer, any sarcoma, including but not limited to fibrosarcoma, angiosarcoma, adenocarcinoma, and rhabdomyosarcoma, and any blood cancer, including all types of leukemia, lymphoma, or myeloma. In embodiments, the disclosure comprises selecting an individual who has been diagnosed with cancer and administering a composition comprising one or more described OVs to the individual. The method may further comprise testing the individual to determine the efficacy of the described therapy, e.g., monitoring the status of the cancer in the individual over a period of time subsequent to, or during a dosing regimen. In embodiments, a composition described herein is administered to an individual who previously had cancer, or is at risk for developing cancer, and thus prophylactic approaches are included by this disclosure. In embodiments, administration of a composition comprising OVs stimulates an anti-cancer immune response, or another response that potentiates an anti-cancer immune response. In embodiments, administration of OVs initiates immunogenic cell death (ICD), releases damage-associated molecular patterns (DAMPs), virus-derived pathogen-associated molecular patterns (PAMPs), and combinations thereof.

In embodiments, a composition of the disclosure is combined with another anti-cancer therapy. In embodiments, a composition of the disclosure is administered concurrently or sequentially with a chemotherapeutic agent. In embodiments, the one or more OVs may potentiate the effect of a co-administered anti-cancer agent. Thus, the disclosure includes synergistic approaches. In embodiments, the chemotherapeutic agent is one or a combination of Doxorubicin (Adriamycin), Cisplatin, Cyclophosphamide, Carboplatin, Pegylated Liposomal Doxorubicin, Methotrexate, Paclitaxel, Fluorouracil, Docetaxel, Liposomal Doxorubicin, Gemcitabine, Cyclophosphamide Irinotecan, or Flutamide. In embodiments, a described composition is administered in combination with one or more checkpoint inhibitors. In embodiments, the checkpoint inhibitor comprises an anti-programmed cell death protein 1 (anti-PD-1) checkpoint inhibitor, or an anti-Cytotoxic T-lymphocyte-associated protein 4 (anti-CTLA-4) checkpoint inhibitor, and agents that bind to the ligands of these checkpoint proteins, such as anti-PD-L1 agents. There are numerous such checkpoint inhibitors known in the art. For example, anti-PD-1 agents include Pembrolizumab and Nivolumab. An anti-PD-L1 example is Avelumab. An anti-CTLA-4 example is Ipilimumab. The described approaches may also be combined with other immunotherapies, such as CAR T cell therapies, radiation, surgical interventions, and the like.

The disclosure also includes articles of manufacture and kits comprising pharmaceutical compositions and/or isolated OVs as described herein. The article of manufacture may include printed material, such as a label, that provides an indication that the pharmaceutical composition and/or isolated OVs are for use in treating cancer. The article of manufacture may include one or more sealed containers such as vials that contain the pharmaceutical compositions and/or isolated OVs.

Also provided are kits for making the described OVs. The kits comprise one or more expression vectors for use in making the described OVs. The kits may further comprise suitable cells for modifying and producing the described OVs, representative examples of which include CHSE (salmon) cells for making or modifying OV1 or chicken embryo fibroblast (CEFs) for making or modifying OV2, and combinations thereof.

The following Examples are intended to illustrate but not limit the disclosure.

### Example 1

This Example demonstrates that the described OVs exhibit anti-cancer activity against a variety of cancer types in vitro. In this regard, we tested the OVs for their oncotoxic property using a range of cancer cells including human lung carcinoma (A549), renal cell adenocarcinoma (769P), kidney clear cell carcinoma (CaKi-2), colon carcinoma (HCT116), colorectal adenocarcinoma (Caco-2, HRT-18G), hepatocellular carcinoma (Huh7.5, C3A), pancreatic adenocarcinoma (BxPC3), mammary ductal carcinoma cells (HCC1500 and HCC1187), leukemia (THP-1), and lymphoma (U937). Oncoselectivity, the ability to selectively target cancer cells and spare healthy spare cells, is a crucial feature of ideal OVs. We infected a range of non-cancerous cells, including human primary bronchial epithelial cells, embryonic kidney cells (293T), fetal brain microglial cells (CHME3), and fetal lung fibroblasts (MRC-5) with OV1 or OV2. None of these cells show any significant cell death or activation of apoptosis, thereby establishing the oncoselectively of OV1 and OV2.

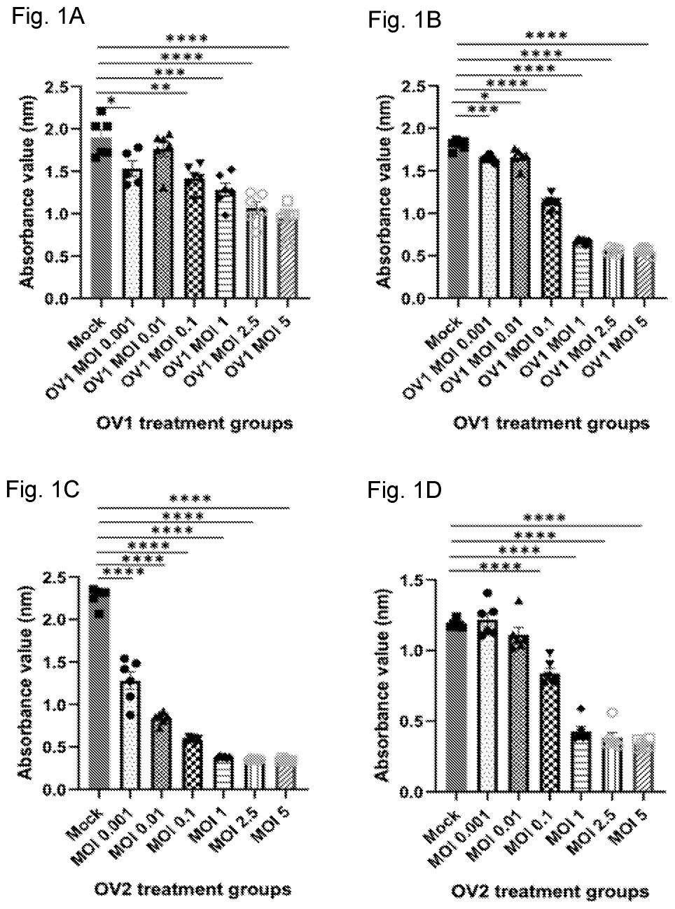

OV1 and OV2 kills a wide range of cancer cells: We evaluated oncotoxicity, which is the ability to infect and cause the death of cancer cells of OV1 and OV2. To evaluate the oncotoxicity of OV1 and OV2, we measured mitochondrial metabolic rate using MTS (3-(4,5-dimethylthiazol-2-yl)-5-(3-carboxymethoxyphenyl)-2-(4-sulfophenyl)-2H-tetrazolium) assay. MTS assay indirectly reflects viable cell numbers and is widely used to quantify cytotoxicity. The viability of renal adenocarcinoma cells (769P) and mammary ductal carcinoma cells (HCC1187) significantly decreased at 72 hours after infection with OV1 (FIGS. 1A & 1B) or OV2 (FIGS. 1C & 1D). Notably OV1 and OV2 significantly reduced viability of 769P and HCC1187 even at very low doses, i.e., multiplicity of infection (MOI) of 0.001 (* p<0.05, ** p<0.01, *** p<0.001, **** p<0.0001).

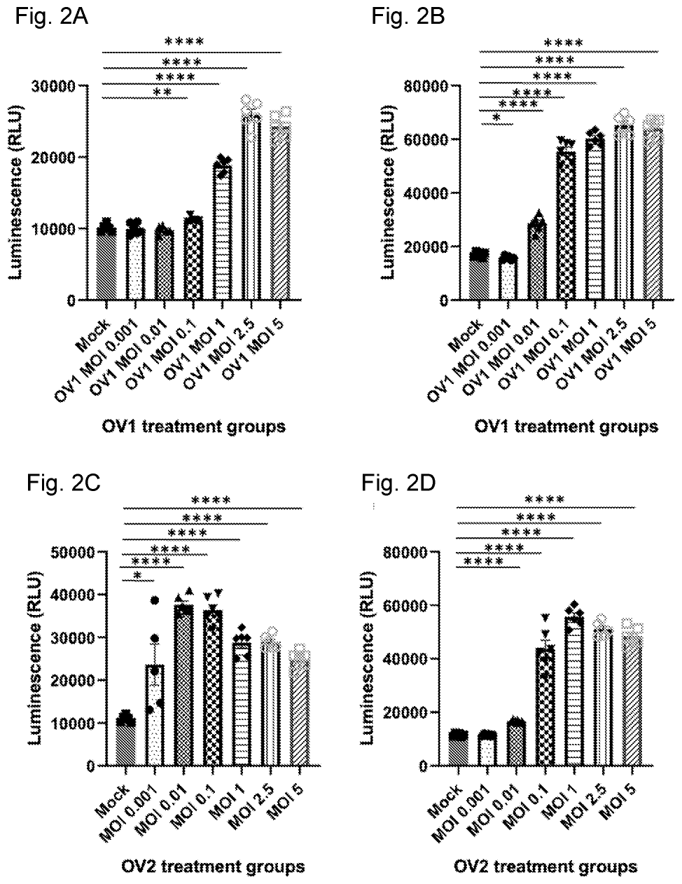

OV1 and OV2 activate apoptotic death of cancer cells. Evasion of apoptosis, also known as programmed cell death, is one of the hallmarks of cancer cells. Therefore, triggering apoptosis is an effective way to kill cancer cells. Caspases are central components of the machinery responsible for apoptosis. Caspases 3 and 7 are the effector caspases responsible for the proteolytic cleavage of a broad spectrum of cellular targets, leading to cell death. We measured activated levels of caspase 3 and 7 using Caspase-Glo® 3/7 assay (Promega). Apoptosis levels as measured by the levels of activated Caspase 3 and 7 in renal adenocarcinoma cells (769P) (MOI≥0.001) and mammary ductal carcinoma cells (HCC1187) (MOI≥0.01) increased significantly at 72 hours after infection with OV1 (FIGS. 2A & 2B) or OV2 (FIGS. 2C & 2D); (* p<0.05, ** p<0.01, *** p<0.001, **** p<0.0001).

### Example 2

This Example demonstrates that the described OVs exhibit anti-cancer activity in a clinically relevant model of breast cancer.

After establishing the oncoselectivity and oncotoxicity of OV1 and OV2 in vitro, we then used 4T1.2 mouse model to evaluate the efficacy of OV1 and OV2 on tumor growth and regression in vivo. As is known in the art, the 4T1 orthotopic breast cancer cell line is a weakly immunogenic, metastatic tumor model when inoculated into the mammary gland of syngeneic BALB/c mice that models triple negative breast cancer in humans¹⁷. Data is this disclosure were obtained using a clone of the parental 4T1 line, 4T1.2. These cells have a high tendency to metastasize to bone¹⁸, a major site of metastasis in humans.

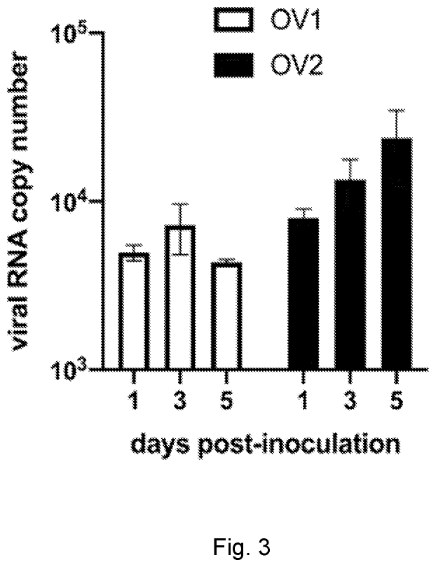

To produce the data discussed below, we first cultured the 4T1.2 cells and infected them with OV1 and OV2 in vitro to demonstrate that both the viruses replicate in these cells (FIG. 3). To produce these data, 4T1.2 cells were infected with OV1 or OV2 for 2 hours using the multiplicity of infection (MOI) of 0.1 and cells were incubated at 37° C. Triplicate wells for each virus treatment was used and cell culture supernatants harvested after 1, 3 and 5 days of infection were used for viral RNA extraction. Extracted viral RNA was analyzed for quantification of viral RNA copy number using a one-step qRT-PCR assay. The results are shown in FIG. 3.

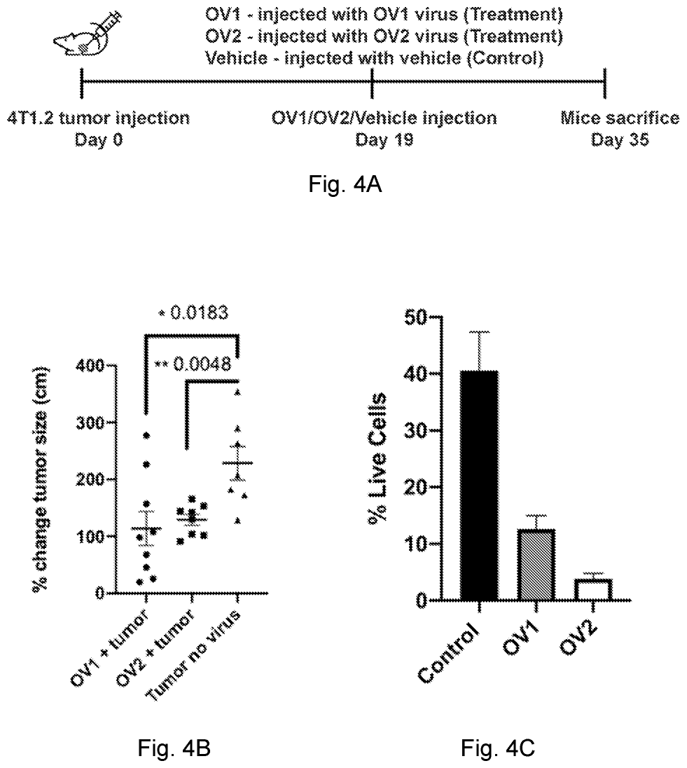

Sixty 8-week-old, female BALB/c mice (obtained from Jackson Laboratory) were group housed, 5 to a cage, in the Centralized Biological Laboratory (CBL) at Pennsylvania State University. Five days after arrival, 40 mice were injected with 5×10⁴ 4T1.2 cells resuspended in 50 uL sterile PBS in the 4^{th} mammary gland on the left side. Tumor implantation day was considered day 0. 20 mice, two groups of 10, were not implanted with 4T1.2 cells. These mice received intraperitoneal virus injections to mirror the injection received by mice with tumors. After tumor injection, mice were weighed, and tumors were palpated/measured with electric calipers twice weekly. The tumors were first palpable in some mice starting around day 10 post tumor implantation, and palpable in most mice by day 12. On day 19, all mice were injected with 50 μL of OV1 or OV2, diluted to 1×10⁵ TCID₅₀ units per 50 μL or 50 μL of virus suspension media for negative control mice. Mice with tumors received 25 μL (half dose) directly into the tumor and 25 μL (half dose) intraperitoneal (IP). Non-tumor control mice received the full volume IP. 15 mice received the OV1 or OV2 treatment and 10 mice received the vehicle control. Tumor size was monitored 1 day prior and day 18 post OV or control injections (FIG. 4A). The percent change in the tumor size from day 1 to day 18 in mice that received OV1 and OV2 is markedly smaller than untreated mice (FIG. 4B). On day 35 post tumor injection, all surviving mice were sacrificed and tumor, blood, both lungs and spleen were harvested. 11 tumors (3 OV2, 4 OV1 and 4 vehicle) were divided and processed and analyzed via flow cytometry. Flow cytometry data was gated and analyzed using FlowJo and plotted in graph pad as treatment means with SEM. Percent live cells were markedly reduced in OV1 and OV2 treated tumors compared to the controls (FIG. 4C).

### Example 3

This example provides a non-limiting description of modifications made to the described OVs, such as engineering the OVs to encode and express therapeutic payloads, and a prophetic description of testing modified OVs in animal models. In this regard, therapeutic payloads are expressed selectively in cancer cells during replication, resulting in complementary mechanism of actions (MOAs). Non-limiting examples of therapeutic payloads are described above. For this Example, the payloads include hGM-CSF¹⁹⁻²⁴ and sodium iodide symporter gene²⁵⁻²⁸. Different OVs relative to those described herein express a transgene for p53 (TP53) or another p53 family member (TP63 or TP73) to generate more potent OVs that function synergistically with host immunity²⁹. Herpes simplex virus type-1 (HSV1) with its neurovirulence factor ICP34.5 inactivated has been shown to direct tumor-specific cell lysis in several tumor models and was shown to be safe in Phase I clinical trials by intra-tumoral injection in glioma and melanoma patients³⁰ Thus, the present disclosure includes modifying the presently described novel OVs to express one or more therapeutic payloads.

The disclosure includes generating recombinant OV1 and OV2 using a well-established reverse genetics system³¹. Viral protein VP5 is initiated at the second in-frame start codon and is dispensable for OV1 as the deletion of VP5 does not affect virus replication nor the apoptosis of infected cells³²⁻³³. In addition, VP5 is the protein is involved in non-lytic egression of virus particles from cells¹⁵. The rationale for creating recombinant OV1 and OV2 by suppressing the expression of viral protein VP5 is that these viruses will only exit the cells by cytolytic pathway which will further enhance the oncolytic potential of these viruses. Full-length cDNA clones of the OV1 genome RNA segments A and B are constructed using NEB HiFi DNA assembly kit. Using genomic RNA as a template, overlapping 3 cDNA fragments each of segment A and B are synthesized and amplified using standard techniques. Destination vectors with the inserts extracted from transformed bacterial cells are used as templates for in vitro transcription with T7 RNA polymerase. cRNAs are transfected either in CHSE cells (for OV1) of chicken embryo fibroblast (CEFs) for OV1. To characterize recovered viruses, either RT-PCR on total nucleic acids isolated from IPNV-infected CHSE cells or immunofluorescence is performed. By using the reverse genetics system for OV1 and OV2, clones of modified segment A with mouse or human Granulocyte-macrophage colony-stimulating factor (GM-CSF) replacing VP5 sequence and full segment B of OV1 or OV2 is generated. Using the same process described above cRNAs and rescue recombinant OV1 or OV2 carrying mouse or human GM-CSF in appropriate cell culture systems (CHSE for OV1 and CEF for OV2) are generated.

In this disclosure, using synthetic DNA and reverse genetics, the following constructs were produced: IPNV Δ VP-5 (IPN virus that does not produce the VP5 protein); IPNV-GM-CSF (IPN virus that expresses GM-CSF); and IPNV Δ VP-5-GM-CSF (a VP5 deficient IPN virus that expresses GM-CSF).

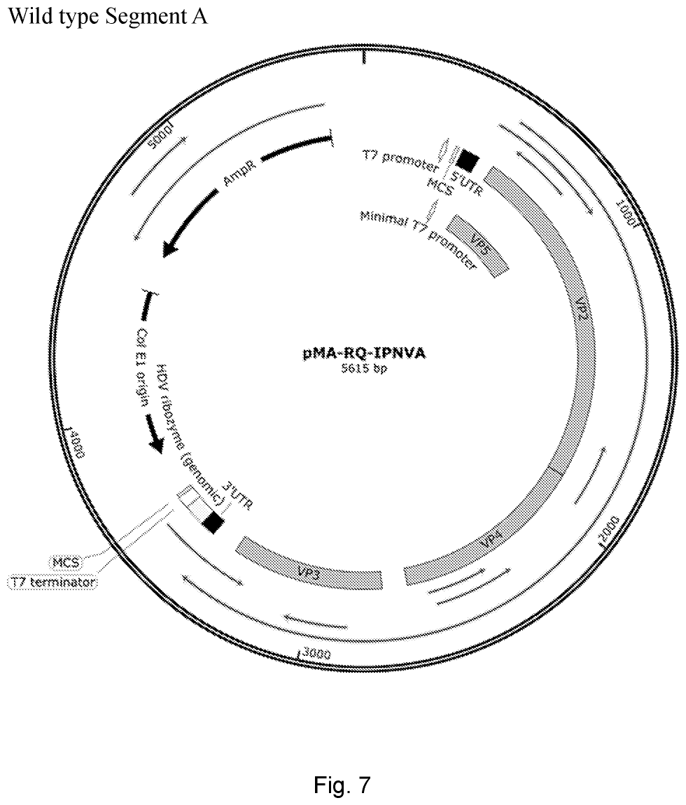

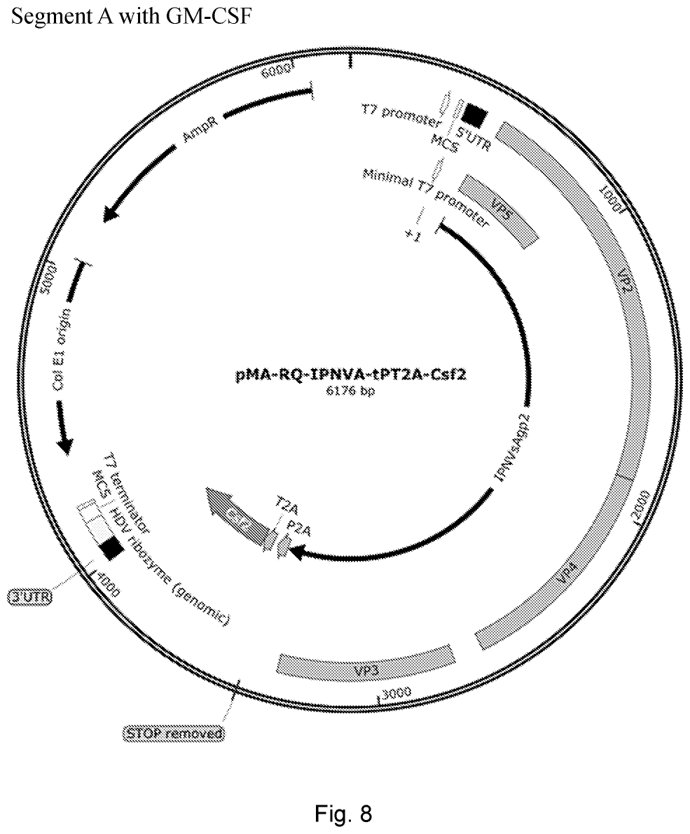

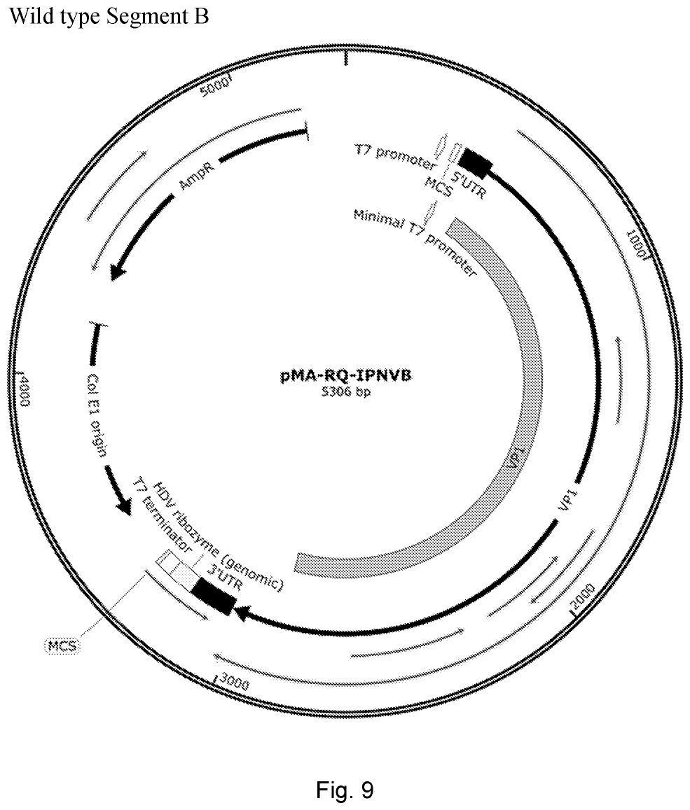

These constructs as produced are demonstrated using the constructs shown in FIGS. 7, 8 and 9.

In an additional embodiment, an engineered virus that does not produce the VP5 protein was produced by inserting a mutation in the following sequence:

(SEQ ID NO: 9)
AGTGGTAACCCACGAGCGGAGAGCTCTTACGGAGGAGCTCTC
CGTCGATGGCGAAAGCCCTTTCTAACAAACAACCAACAATTC
TTATCTACAGGAATCATGAGCACATCCAAGGCAACCGCAACC
TACTTGAGATCCATTATGCTTCCCGAGAATGGGCCAGCAAGC
ATTCCGGATGACATAACAGAGAGGCATATACTAAAACAAGAG
ACCTCGTCATACAACTTAG.

The positions of the mutations are shown in bold and italics. The G removes the ATG of VP5 (ATG→AGG). The second mutation T introduces a STOP codon in the reading frame of VP5 (Arg→STOP) while keeping the Asp in the reading frame of VP2 intact.

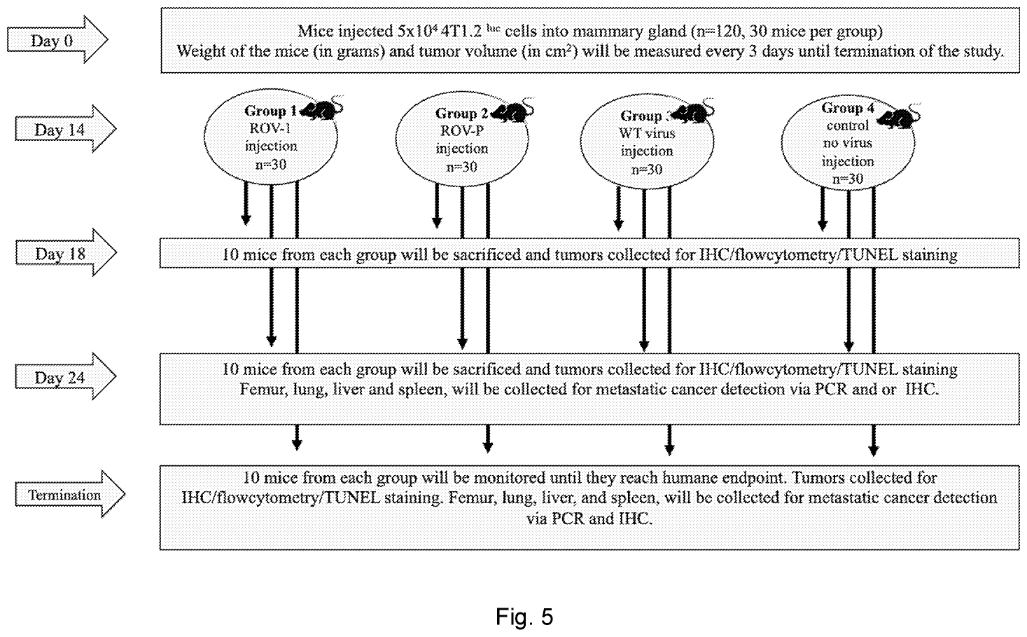

The modified OVs described above are tested in an animal model of cancer, such as a 4T1 mouse model as generally depicted in FIG. 5. 120 female BALB/c mice are injected with 5×10⁴ 4T1.2^{luc} in the right flank mammary gland, at 12 weeks of age, to induce tumor formation. 14 days after tumor cell injection, palpated, measured tumors are intratumorally injected with 25 μl of virus (or control) in one of four treatment groups, Group 1: Recombinant OV1/OV2 virus: ROV-1, Group 2: Recombinant OV1/OV2 virus+payload protein: ROV-P, Group 3: OV1/OV2 WT virus, Group 4: No virus control (isotonic solution injection), n=30 per group. Four days post infection (DPI) 10 mice from each group are sacrificed and tumors are collected for immunohistochemistry to check for viral antigen, flow cytometry and terminal deoxynucleotidyl transferase dUTP nick end labeling (TUNEL) assay to detect DNA breaks formed during the final phase of apoptosis. On day 24, (10 DPI), 10 mice from each group are sacrificed and tumors collected for IHC/flow cytometry/TUNEL signaling. Femur, lung, liver, and spleen, are collected for metastatic cancer detection using PCR and IHC. The 4T1.2 cells have been engineered to stably express luciferase (luc) for ease of quantification of metastatic burden. We have used these cells in previous experiments to characterize primary tumor growth, metastasis and immune outcomes³⁴. PCR for luciferase is used to detect metastasis in tissue. The final 10 mice in each group are carried forward to measure if virus treatments extend survival in treated mice. All mice are sacrificed when predetermined endpoints have been reached, which include tumor size exceeding 1.5 cm², or significant signs of illness (weight change) or impairments (movement restricted by tumor growth). Tumors are collected for IHC/flow cytometry/tunnel signaling. Femur, lung, liver, and spleen, are collected for metastatic cancer detection via PCR and IHC at an endpoint or not more than 35 days post tumor cell injection. IHC is performed on 5-micron paraffin sections from tumors and tissues collected for GFP signaling and immunostaining for viral antigen using specific antibodies against OV1 or OV2. Images are collected on an inverted REVOLVE ECHO microscope. TUNEL assay is performed in combination with IHC to co-localize signal for cell apoptosis and viral infection.

Immune outcomes: Spleens and tumors collected from mice at sacrifice are used to assess immune outcomes. Splenic CD4+ T cells are purified from tumor-bearing mice using magnetic bead depletion, stimulated with increasing concentrations of anti-CD3 antibodies (0-1 μg/mL), and proliferative capacity will be assessed by DNA synthesis (tritiated thymidine uptake). In addition, splenocytes from tumor bearing mice are cultured in vitro with irradiated 4T1.2luc tumor cells to induce tumor antigen-induced IFN-γ production. In addition to the assessment of splenic T cell function in the experiments outlined above, tumor infiltrating lymphocytes (TILs) (NK cells, CD4+ and CD8+ T cells); inflammatory infiltrates (macrophages); and MDSCs are assessed in the tumor in mice (n=7/group) via flow cytometry. These same populations are assessed in the spleen to evaluate the effect of treatment on the distribution of immune cells in the periphery. Single cell suspensions will be made from the tumors and spleens. CD3⁺/CD4⁺, CD3⁺/CD8⁺, NK1.1⁺/CD3⁺, CD11b⁺/F480⁺, and Gr-1⁺/CD11b⁺ antibodies are used to identify T cells subsets, NK cells, macrophages, and MDSCs, respectively. In a subset of mice (n=3/group) the tumors are frozen, homogenized, and RNA prepared for use in the Cancer Inflammation & Immunity Crosstalk PCR Array (Qiagen, www.sabiosciences.com/rt_pcr_product/HTML/PAMM-181Z.html) which profiles the expression of 84 key genes involved in mediating communication between tumor cells and the cellular mediators of inflammation and immunity.

### Example 4

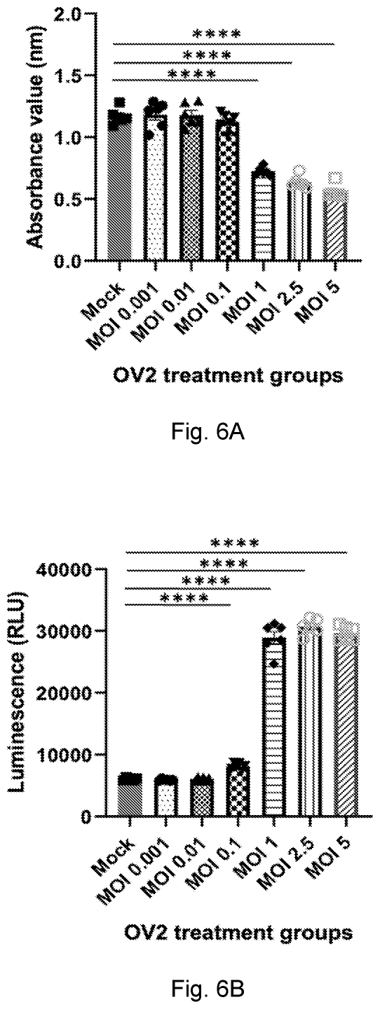

This example demonstrates that the described OVs exhibit anti-cancer activity against canine (Canis familiaris) cancer cells in vitro and, therefore, possess potential therapeutic capabilities for veterinary cancer. We tested the OVs for oncotoxic activity using canine mammary carcinoma cells (CMT-U27). To test for oncoselectivity, we used a non-cancerous canine renal epithelial cell (MDCK) as a control. The cells were infected with OV2 at MOIs of 0.001, 0.01, 0.1, 1, 2.5, and 5. At 72 hours post-infection, the viability of cells was tested using an MTS assay, and cell apoptosis was quantified using Caspase-Glo 3,7 assay. OV2 infection resulted in a significant decline in viability (FIG. 6A) and increased apoptosis (FIG. 6B). No such effects were noticed in the control MDCK cells.

The following reference listing is not an indication that any of the references are material to patentability.

### REFERENCES

1 Fukuhara, H., Ino, Y. & Todo, T. Oncolytic virus therapy: A new era of cancer treatment at dawn. Cancer Sci 107, 1373-1379, doi: 10.1111/cas.13027 (2016).
2 Zamarin, D. & Palese, P. Oncolytic Newcastle disease virus for cancer therapy: old challenges and new directions. Future Microbiol 7, 347-367, doi: 10.2217/fmb.12.4 (2012).
3 Li, P. et al. Therapeutic effects of a fusogenic newcastle disease virus in treating head and neck cancer. Head Neck 33, 1394-1399, doi: 10.1002/hed.21609 (2011).
4 Mansour, M., Palese, P. & Zamarin, D. Oncolytic specificity of Newcastle disease virus is mediated by selectivity for apoptosis-resistant cells. J Virol 85, 6015-6023, doi:10.1128/JVI.01537-10 (2011).
5 Silberhumer, G. R. et al. Genetically engineered oncolytic Newcastle disease virus effectively induces sustained remission of malignant pleural mesothelioma. Mol Cancer Ther 9, 2761-2769, doi: 10.1158/1535-7163.MCT-10-0090 (2010).
6 Song, K. Y. et al. Antitumor efficacy of viral therapy using genetically engineered Newcastle disease virus [NDV(F3aa)-GFP] for peritoneally disseminated gastric cancer. J Mol Med (Berl) 88, 589-596, doi: 10.1007/s00109-010-0605-6 (2010).
7 Zamarin, D., Vigil, A., Kelly, K., Garcia-Sastre, A. & Fong, Y. Genetically engineered Newcastle disease virus for malignant melanoma therapy. Gene Ther 16, 796-804, doi: 10.1038/gt.2009.14 (2009).
8 Hastie, E. & Grdzelishvili, V. Z. Vesicular stomatitis virus as a flexible platform for oncolytic virotherapy against cancer. J Gen Virol 93, 2529-2545, doi: 10.1099/vir.0.046672-0 (2012).
9 Koske, I. et al. Oncolytic virotherapy enhances the efficacy of a cancer vaccine by modulating the tumor microenvironment. Int J Cancer 145, 1958-1969, doi:10.1002/ijc.32325 (2019).
10 Schreiber, L. M. et al. The lytic activity of VSV-GP treatment dominates the therapeutic effects in a syngeneic model of lung cancer. Br J Cancer 121, 647-658, doi: 10.1038/s41416-019-0574-7 (2019).
11 Bishnoi, S., Tiwari, R., Gupta, S., Byrareddy, S. N. & Nayak, D. Oncotargeting by Vesicular Stomatitis Virus (VSV): Advances in Cancer Therapy. Viruses 10, doi: 10.3390/v10020090 (2018).
12 Kimpel, J. et al. The Oncolytic Virus VSV-GP Is Effective against Malignant Melanoma. Viruses 10, doi: 10.3390/v10030108 (2018).
13 Urbiola, C. et al. Oncolytic activity of the rhabdovirus VSV-GP against prostate cancer. Int J Cancer 143, 1786-1796, doi: 10.1002/ijc.31556 (2018).
14 Durham, N. M. et al. Oncolytic VSV Primes Differential Responses to Immuno-oncology Therapy. Mol Ther 25, 1917-1932, doi: 10.1016/j.ymthe.2017.05.006 (2017).
15 Gimenez, M. C., Zanetti, F. A., Terebiznik, M. R., Colombo, M. I. & Delgui, L. R. Infectious Bursal Disease Virus Hijacks Endosomal Membranes as the Scaffolding Structure for Viral Replication. J Virol 92, doi: 10.1128/JVI.01964-17 (2018).
16 Gantier, M. P. & Williams, B. R. The response of mammalian cells to double-stranded RNA. Cytokine Growth Factor Rev 18, 363-371, doi:10.1016/j.cytogfr.2007.06.016 (2007).
17 Aslakson, C. J. & Miller, F. R. Selective events in the metastatic process defined by analysis of the sequential dissemination of subpopulations of a mouse mammary tumor. Cancer Res 52, 1399-1405 (1992).
18 Eckhardt, B. L. et al. Genomic analysis of a spontaneous model of breast cancer metastasis to bone reveals a role for the extracellular matrix. Mol Cancer Res 3, 1-13, doi:3/1/1 [pii] (2005).
19 Malhotra, S. et al. Use of an oncolytic virus secreting GM-CSF as combined oncolytic and immunotherapy for treatment of colorectal and hepatic adenocarcinomas. Surgery 141, 520-529, doi:10.1016/j.surg.2006.10.010 (2007).
20 Giacomantonio, M. A. et al. Quantitative Proteome Responses to Oncolytic Reovirus in GM-CSF- and M-CSF-Differentiated Bone Marrow-Derived Cells. J Proteome Res, doi: 10.1021/acs.jproteome.9b00583 (2020).
21 Goins, W. F. et al. Engineering HSV-1 Vectors for Gene Therapy. Methods Mol Biol 2060, 73-90, doi: 10.1007/978-1-4939-9814-2_4 (2020).
22 Kemp, V. et al. Arming oncolytic reovirus with GM-CSF gene to enhance immunity. Cancer Gene Ther 26, 268-281, doi: 10.1038/s41417-018-0063-9 (2019).
23 Penghui, Y. et al. Oncolytic Activity of a Novel Influenza A Virus Carrying Granulocyte-Macrophage Colony-Stimulating Factor in Hepatocellular Carcinoma. Hum Gene Ther 30, 330-338, doi: 10.1089/hum.2018.095 (2019).
24 Zhuang, X. F. et al. [The therapeutic effect of HSV1-hGM-CSF combined with doxorubicin on the mouse breast cancer model]. Zhonghua Zhong Liu Za Zhi 40, 178-185, doi: 10.3760/cma.j.issn.0253-3766.2018.03.004 (2018).
25 Dispenzieri, A. et al. Phase I trial of systemic administration of Edmonston strain of measles virus genetically engineered to express the sodium iodide symporter in patients with recurrent or refractory multiple myeloma. Leukemia 31, 2791-2798, doi: 10.1038/leu.2017.120 (2017).
26 Wang, J. et al. Enhancing Expression of Functional Human Sodium Iodide Symporter and Somatostatin Receptor in Recombinant Oncolytic Vaccinia Virus for In Vivo Imaging of Tumors. J Nucl Med 58, 221-227, doi: 10.2967/jnumed. 116.180463 (2017).
27 Mansfield, D. C. et al. Oncolytic vaccinia virus as a vector for therapeutic sodium iodide symporter gene therapy in prostate cancer. Gene Ther 23, 357-368, doi:10.1038/gt.2016.5 (2016).
28 Miller, A. & Russell, S. J. The use of the NIS reporter gene for optimizing oncolytic virotherapy. Expert Opin Biol Ther 16, 15-32, doi: 10.1517/14712598.2016.1100162 (2016).
29 Bressy, C., Hastie, E. & Grdzelishvili, V. Z. Combining Oncolytic Virotherapy with p53 Tumor Suppressor Gene Therapy. Mol Ther Oncolytics 5, 20-40, doi:10.1016/j.omto.2017.03.002 (2017).
30 Liu, B. L. et al. ICP34.5 deleted herpes simplex virus with enhanced oncolytic, immune stimulating, and anti-tumour properties. Gene Ther 10, 292-303, doi:10.1038/sj.gt.3301885 (2003).
31 Yao, K. & Vakharia, V. N. Generation of infectious pancreatic necrosis virus from cloned cDNA. J Virol 72, 8913-8920, doi: 10.1128/JVI.72.11.8913-8920.1998 (1998).
32 Santi, N., Song, H., Vakharia, V. N. & Evensen, O. Infectious pancreatic necrosis virus VP5 is dispensable for virulence and persistence. J Virol 79, 9206-9216, doi:10.1128/JVI.79.14.9206-9216.2005 (2005).
33 Santi, N. et al. Infectious pancreatic necrosis virus induces apoptosis in vitro and in vivo independent of VP5 expression. Virology 342, 13-25, doi:10.1016/j.virol.2005.07.028 (2005).
34 Turbitt, W. J. et al. Physical Activity Plus Energy Restriction Prevents 4T1.2 Mammary Tumor Progression, MDSC Accumulation, and an Immunosuppressive Tumor Microenvironment. Cancer Prev Res (Phila) 12, 493-506, doi: 10.1158/1940-6207.CAPR-17-0233 (2019).

1. A method for treatment or prophylaxis of cancer in an individual in need thereof comprising administering an effective amount of an oncolytic double stranded RNA (dsRNA) virus (“OV”) to the individual, wherein the dsRNA virus is selected from Infectious Pancreatic Necrosis virus (“OV1”) and Infectious Bursal disease Virus (“OV2”), wherein the OV1 or the OV2 optionally comprises a genetic modification.

2. The method of claim 1, wherein the OV1 or the OV2 comprises a genetic modification of its genome that optionally enhances its oncolytic function and/or retains its oncolytic function at a temperature that is lower than a temperature of a natural host of the OV1 or the OV2.

3. The method of claim 2, wherein the OV1 or the OV2 comprises the genetic modification that enhances its oncolytic function relative the oncolytic function of the an unmodified OV1 or OV2.

4. The method of claim 3, wherein the genetic modification comprises a sequence encoding a therapeutic payload.

5. The method of claim 4, wherein the therapeutic payload comprises Granulocyte-macrophage colony-stimulating factor.

6. The method of claim 2, wherein the genetic modification comprises a disruption or mutation of a segment of the viral genome that encodes the viral VP5 protein such that the viral VP5 protein is not produced within cells infected with the OV1 or the OV2.

7. The method of claim 1, wherein the individual in need thereof is a mammal that is optionally a human or a canine.

8. The method of claim 7, wherein administering the OV1 or the OV2 inhibits growth of cancer cells in the individual.

9. The method of claim 8, wherein the cancer cells are present in a tumor.

10. The method of claim 7, comprising administering an effective amount of the OV1.

11. The method of claim 7, comprising administering an effective amount of the OV2.

12. A cDNA amplified from a segment of a genomic RNA of an OV of claim 1.

13. A cRNA transcribed from a cDNA of claim 12.

14. An isolated or recombinantly produced oncolytic double stranded RNA (dsRNA) virus (“OV”) for use in prophylaxis or therapy of cancer, wherein the OVs are selected from a Birnaviridae aquabirnavirus that is Infectious Pancreatic Necrosis (IPN) virus (IPNV) (OV1) or a modified OV1, a Birnaviridae avibirnavirus which is poultry virus Infectious Bursal disease Virus (IBDV) (OV2) or a modified OV2.

15. The isolated OV of claim 14, wherein the OV comprises a genetic modification.

16. The OV of claim 15, wherein the genetic modification comprises a disruption or mutation of a segment of the viral genome that encodes the viral VP5 protein such that the viral VP5 protein is not produced within cells infected with the OV1 or the OV2.

17. The OV of claim 16, wherein the genetic modification comprises a sequence encoding a therapeutic payload.

18. The OV of claim 16, wherein the therapeutic payload comprises Granulocyte-macrophage colony-stimulating factor.

19. Cancer cells comprising an isolated or recombinantly produced OV of claim 14.

20. A pharmaceutical composition comprising an OV of claim 14.

21. One or more expression vectors encoding one or two segments of an OV of claim 14.
